(19) United States
(12) Patent Application Publication
(10) Pub. No.: US 2007/0161827 A1
(43) Pub. Date: Jul. 12, 2007

(54) Method of synthesizing fluorinated diene alcohols

(75) Inventors: Jingji Ma, West Seneca, NY (US); Michael Van Der Puy, Amherst, NY (US); David Nalewajek, West Seneca, NY (US); Haridasan Nair, Williamsville, NY (US); Lawrence Ford, Hamburg, NY (US)

Correspondence Address: HONEYWELL INTERNATIONAL INC., 101 COLUMBIA ROAD, P O BOX 2245, MORRISTOWN, NJ, 07962-2245, US

(73) Assignee: Honeywell International Inc., Morristown, NJ (US)

(21) Appl. No.: 11/328,048

(22) Filed: Jan. 9, 2006

(51) Int. Cl. C07C 33/42

(52) U.S. Cl. 568/843

(57) ABSTRACT

Disclosed are fluorinated diene alcohols perferably having a fluorine content of at least about 45 weight percent and having a plurality of moieties of the formula CFₓ and a plurality of moieties of the formula CHₓ, where each x is independently 1, 2 or 3 and where each CFₓ moiety is not directly bonded to another CFₓ moiety. Methods of synthesizing fluorinated diene alcohol compounds are also disclosed comprising reacting at least one C₂-C₃ alkene halide with at least one reactant of the formula (II): 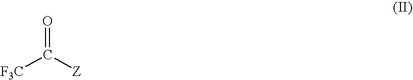
where Z is OH, OR⁶, OC(O)R⁷, or a halogen,
R⁶ is a C₁-C₆ branched or straight chain alkyl, and
R⁷ is a C₁-C₆ branched or straight chain alkyl or fluoroalkyl,

### BACKGROUND

(1) Field of Invention

The present invention relates to fluorinated diene alcohols and to methods for synthesizing same.

(2) Description of Related Art

Certain fluorinated diene alcohols are known in the art. For example, U.S. Pat. No. 6,858,692 (Kaneko) describes fluorinated diene alcohols including CF₂═CFCF₂C(OH)(CF₃)CH═CH₂ and CF₂═CFCF₂C(OH)(CF₃)CH₂CH═CH₂. The synthesis techniques described by Kaneko for these compounds are complex and involve multiple processing steps. Homopolymers and copolymers of these fluorinated diene alcohols were studied as materials for photoresists at 157 nm, since research in the field has shown that fluorinated substituents reduce the absorption of various polymers at 157 nm (Marcromolecules 2002, 35, 6539).

Thus, there remains a need for novel fluorinated diene alcohols having low 157 nm absorption coefficients, as well as simple and economic methods for producing such compounds. The present invention satisfies these and other needs in the art.

### SUMMARY OF THE INVENTION

The present invention relates generally to novel fluorinated diene alcohols, and to methods in general for synthesizing fluorinated diene alcohols, including the novel compounds of the present invention. The fluorinated diene alcohols may be symmetrical or asymmetrical and in preferred embodiments are formed from C₂-C₃ alkene halides. In preferred embodiments, the fluorinated diene alcohols made by the present methods, and the novel fluorinated diene alcohols in particular, have relatively low 157 nm absorption coefficients. The fluorinated diene alcohols of the present invention also perferably have a fluorine content of at least about 45 weight percent and have a plurality of ═CF—, ═CF₂, —CF₂—, and/or —CF₃ moieties, wherein substantially each of these moieties, and even more preferably each such moiety, is separated from each other such moiety by at least one carbon atom that is not bonded to a fluorine atom. Applicants have discovered that the 157 nm absorption coefficient of such compounds in general is lower than the absorption coefficient of fluorinated diene alcohols having the same fluorine content but in which two or more the above-mentioned fluorinated moieties bonded direct to each other.

One aspect of the present invention therefore provides fluorinated diene alcohol compounds comprising at least about 45 percent by weight of fluorine and having: from about 6 to about 11 carbon atoms; a plurality of moieties of the formula CFₓ; and a plurality of moieties of the formula CHₓ, wherein each x is independently 1, 2 or 3 and wherein each CFₓ moiety is not directly bonded to another CFₓ moiety. Particularly preferred embodiments of this aspect of the invention are fluorinated diene alcohols having the formula (I) below: 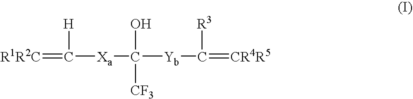
wherein R¹, R², R⁴, and R⁵ are each independently H or F; R³ is H, CH₃, or CF₃; X and Y are independently CH₂ or CF₂; and a and b are independently 0 or 1; provided that if X is CH₂, then at least one of R¹ or R² is fluorine; that if Y is CH₂, then at least one of R³, R⁴, or R⁵ is fluorine or CF₃; and that at least about 45 weight percent of said compound is fluorine.

Another aspect of the present invention provides methods for synthesizing fluorinated diene alcohol compounds comprising reacting, preferably in the presence of metal, at least one C₂-C₃ alkene halide with a compound of the formula (II): 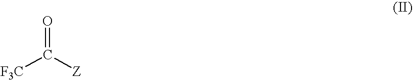
wherein
Z is OH, OR⁶, OC(O)R⁷, or a halogen,
R⁶ is a C₁-C₆ branched or straight chain alkyl, and
R⁷ is a C₁-C₆ branched or straight chain alkyl or fluoroalkyl, to produce a C₆-C₁₁ fluorinated diene alcohol.

### DETAILED DESCRIPTION OF PREFERRED EMBODIMENTS

Compounds of the present invention are preferably fluorinated diene alcohols having: from about 6 to about 11 carbon atoms; a plurality of moieties of the formula CFₓ; and a plurality of moieties of the formula CHₓ, wherein each x is independently 1, 2 or 3 and each CFₓ moiety is not directly bonded to another CFₓ moiety. Preferably said compound comprises at least about 45 percent by weight of fluorine. It is known that the incorporation of fluorinated substituents into photoresist materials reduces the absorption of various structures at 157 nm.

Fluorinated diene alcohols of the present invention preferably have a single —OH group, and can be either symmetrical or asymmetrical with respect to this —OH group. The —OH group is preferably bonded to a carbon atom which, in turn, is bonded to a —CF₃ group to form a —C(OH)(CF₃)— moiety. In certain preferred embodiments, the carbon atom of this —C(OH)(CF₃)— moiety is further bonded to two fluorinated carbon radicals selected from the group consisting of ═CF— and —CF₂—, that is, the compound in such embodiments includes the moiety ═CFC(OH)(CF₃)CF═, or —CF₂C(OH)(CF₃)CF═, or —CF₂C(OH)(CF₃)CF₂—, including all isomers of each of these. The —OH group of these compounds preferably has a pKa value from about 3 to about 7.

In certain other preferred embodiments, the carbon atom of the above-mentioned —C(OH)(CF₃)— moiety is further bonded to one fluorinated carbon selected from the group consisting of ═CF— and —CF₂— and to one hydrogenated carbon selected from the group consisting of ═CH— and —CH₂—. The —OH group of these compounds typically has a pKa value from about 7 to about 10.

In yet other preferred embodiments, the carbon atom of the above-mentioned —C(OH)(CF₃)— moiety is further bonded to two hydrogenated carbons selected from the group consisting of ═CH— and —CH₂—. The —OH group of these compounds typically has a pKa value from about 10 to about 13.

The acidity of the alcohol effects the alcohol's solubility and other reactivity with respect to the resist.

In certain highly preferred embodiments, compounds of the present invention will have the formula (I): 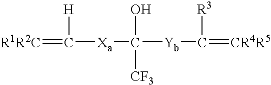
wherein
R¹, R², R⁴, and R⁵ are independently H or F;
R³ is H, CH₃, or CF₃;
X and Y are independently CH₂ or CF₂; and
a and b are independently 0 or 1;
   provided that if X is CH₂, then at least one of R¹ or R² is fluorine; that if Y is CH₂, then at least one of R³, R⁴, or R⁵ is fluorine of CF₃; and that at least about 45 weight percent, more preferably from about 45 to about 70 weight percent, of said compound is fluorine.

Compounds according to this embodiment of the invention may be either symmetrical or asymmetrical. Examples of symmetrical compounds include, but are not limited to, CF₂═CHC(CF₃)(OH)CH═CF₂, CF₂═CHCF₂C(CF₃)(OH)CF₂CH═CF₂, and CH₂═CHCF₂C(CF₃)(OH)CF₂CH═CH₂. Examples of asymmetrical compounds include, but are not limited to, CF₂═CHCF₂C(CF₃)(OH)CH═CH₂, CF₂═CHCF₂C(CF₃)(OH)CH═CF₂, CF₂═CHCF₂C(CF₃)(OH)CF₂CH═CH₂, CH₂═CHCF₂C(CF₃)(OH)CH═CF₂, CH₂═CHCF₂C(CF₃)(OH)C(CH₃)═CF₂, and CH₂═CHCF₂C(CF₃)(OH)CH═CH₂.

For applications involving 157 nm photolithography, fluorinated diene alcohols will preferably have a high weight percentage of fluorine via ═CF—, ═CF₂, —CF₂—, and/or —CF₃ moieties, are separated from each other by at least one carbon atom which is not directly bonded to a fluorine atom. Thus, highly preferred embodiments of the present invention include, but are not limited to, CF₂═CHCF₂C(OH)(CF₃)CF₂C(CF₃)═CF₂, CF₂═CHC(OH)(CF₃)C(CF₃)═CF₂, CF₂═CHC(OH)(CF₃)CH═CF₂, and the like.

According to another aspect of the present invention, provided is a method for synthesizing fluorinated diene alcohols. In certain preferred embodiments, the method comprises reacting, in the presence of metal, a C₂-C₃ alkene halide with a reactant of the formula (II): 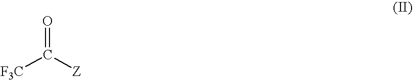
wherein
Z is OH, OR⁶, OC(O)R⁷, or a halogen,
R⁶ is a C₁-C₆ branched or straight chain alkyl, and
R⁷ is a C₁-C₆ branched or straight chain alkyl or fluoroalkyl, to produce a C₆-C₁₁ fluorinated diene alcohol.

In certain preferred embodiments, the C₂-C₃ alkene halide is a vinyl or allyl halide comprising at least one H atom and at least one atom selected from the group consisting of chlorine, bromine, and iodine. Preferably, the C₂-C₃ alkene halide also comprises at least one fluorine atom.

Examples of C₂-C₃ alkene halide for use with the present invention include, but are not limited to, CH₂═CHCH₂Cl, CH₂═CHCF₂Br, CF₂═CHCF₂Br, CH₂═CHBr, CF₂═CHBr, CF₂═C(CH₃)Br, and the like. Many of these compounds are commercially available. For example, allyl chloride is available from Solvay Chemicals, Inc. of Houston, Tex. These C₂-C₃ alkene halide may also be synthesized by various processes. For example, CF₂═CHCF₂Br can by synthesized by first reacting CF₂Br₂ with (PhCO₂)₂ and CF₂═CH₂ to form BrCF₂CH₂CF₂Br, and then reacting the BrCF₂CH₂CF₂Br with NaOH to form the desired CF₂═CHCF₂Br. Likewise, CH₂═CHCF₂Br can by synthesized by first reacting CF₂Br₂ with (PhCO₂)₂ and CH₂═CH₂ to form BrCF₂CH₂CH₂Br, and then reacting the BrCF₂CH₂CH₂Br with NaOH to form the desired CH₂═CHCF₂Br.

In a highly preferred embodiment of the present invention, a simple and economical two-step method of synthesizing an asymmetrical fluorinated diene alcohol is provided comprising: (a) reacting, in the presence of a metal, at least a first C₂-C₃ alkene halide with at least one reactant selected from the group consisting of trifluoroacetic acid, ethyl trifluoroacetate, and trifluoroacetic anhydride to form at least one fluorinated allyl trifluoromethyl ketone; and (b) reacting said allyl trifluoromethyl ketone of step (a) with a second C₂-C₃ alkene halide, preferably in the presence of zinc (such as zinc powder) to form an asymmetrical fluorinated diene alcohol; wherein said first C₂-C₃ alkene and said second C₂-C₃ alkene are different compounds. In certain preferred embodiments, the metal of step (a) is a metal selected from the group consisting of magnesium, zinc, or cadnium. However, it is contemplated that other metals could be used as well.

The first C₂-C₃ alkene halide, metal, and reactant of step (a) are preferably mixed and reacted in the presence of a solvent, such as anhydrous ether or pyridine, at a temperature from about −5° C. to about 25° C., and optionally under a N₂ blanket. After the components are mixed, they are preferably refluxed at an appropriate temperature for approximately 1 to 3 hours. Acid water is preferably then added and the resulting layers are separated and the aqueous phase is extracted. The solvent is then preferably removed from the extract and at least a portion of the remaining residue is distilled to produce product comprising allyl trifluoromethyl ketone.

Examples of reactions to produce the fluorinated allyl trifluoromethyl ketone of step (a) include, but are not limited to:
CF₂═CHCF₂MgBr+CF₃CO₂H→CF₂═CHCF₂COCF₃;
CF₂═CHCF₂Br+CF₃CO₂C₂H₅+Zn→CF₂═CHCF₂COCF₃; and
CH₂═CHCF₂Br+CF₃COCl+Zn→CH₂═CHCF₂COCF₃.

Step (b) is preferably conducted in a manner similar to step (a), but where the allyl trifluoromethyl ketone of step (a), the second C₂-C₃ alkene halide, and zinc powder are mixed instead of the first C₂-C₃ alkene halide, metal catalyst, and reactant.

Examples of step (b) wherein the allyl ketone of step (a) is reacted with a different allyl or vinyl halide in the presence of zinc (preferably zinc powder) to form an asymmetric diene alcohol include, but are not limited to:
CF₂═CHCF₂COCF₃+CH₂═CHBr→CF₂═CHCF₂C(CF₃)(OH)CH═CH₂;
CF₂═CHCF₂COCF₃+CF₂═CHBr→CF₂═CHCF₂C(CF₃)(OH)CH═CF₂;
CF₂═CHCF₂COCF₃+CH₂═CHCF₂Br→CF₂═CHCF₂C(CF₃)(OH)CF₂CH═CH₂;
CH₂═CHCF₂COCF₃+CF₂═CHBr→CH₂═CFCF₂C(CF₃)(OH)CH═CF₂;
CH₂═CHCF₂COCF₃+CF₂═C(CH₃)(Br)→CH₂═CHCF₂C(CF₃)(OH)C(CH₃)═CF₂; and
CH₂═CHCF₂COCF₃+CH₂═CHBr→CH₂═CHCF₂C(CF₃)(OH)CH═CH₂

In another highly preferred embodiment of the present invention, a simple and economical one-step method of synthesizing a symmetrical fluorinated diene alcohol is provided comprising reacting, in the presence of zinc (preferably zinc powder), a C₂-C₃ alkene halide with at least one reactant selected from the group consisting of trifluoroacetic ester and trifluoroacetic anhydride, to form a symmetrical fluorinated diene alcohol.

Preferably, the C₂-C₃ alkene halide and zinc powder are mixed and reacted in the presence of a solvent, such as tetrahydrofuran (THF), at a temperature from about −5° C. to about 25° C. for 4 to 24 hours. The reaction mixture is, preferably, then evaporated (for example at about 25° and about 50 mm Hg) and the fluorinated diene alcohol is collected as a condensate, preferably at a temperature of from about −25° C. to about −95° C. Examples of symmetrical fluorinated diene alcohol synthesized in accordance with this method include, but are not limited to, CF₂═CHC(CF₃)(OH)CH═CF₂, CF₂═CHCF₂C(CF₃)(OH)CF₂CH═CF₂, and CH₂═CHCF₂C(CF₃)(OH)CF₂CH═CH₂.

### EXAMPLES

The following non-limiting examples serve to illustrate certain aspects of the invention.

### Example 1

This Example shows the preparation of the allyl halide, CH₂═CHCF₂Br.

CF₂Br₂ (400 g) and (PhCO₂)₂ (10 g) were charged into a 600 mL autoclave, which was previously purged with N₂ for 30 min. The autoclave was closed and filled with CH₂═CH₂ at 100 psi pressure while stirring. The autoclave was heated to 65° C. and then the heating was switched off. The highest inner temperature was 110° C., then the heating control was set at 90° C. and CH₂═CH₂ pressure was controlled at 200 psi and so maintained for about 24 hours.

The autoclave was then allowed to return to room temperature and excess CH₂═CH₂ was released. From the fractional distillation of the product, excess CF₂Br₂ (230 g) was stripped off and then 120 g BrCH₂CH₂CF₂Br was collected (b.p. 73° C., purity (GC) 97%).

The dehydrobromination was carried out in a 250 mL three neck flask equipped with an addition funnel and a Claison type distillation head. The distillation receiver was cooled in ice. A solution of KOH (120 g) in water (100 ml) was added in the flask and the flask was heated in a 90-100° C. oil bath. BrCH₂CH₂CF₂Br (90 g) was added dropwise from the addition funnel in the flask for about 60 minutes with vigorous stirring. After the addition the reaction was continued at 100-110° C. for 2 hours. 60 g crude product was collected, which was then dried with Na₂SO₄. After fractional distillation, 57 g of product was collected, having a ratio of CH₂═CHCF₂Br to CF₂═CHCH₂Br of 90:10.

### Example 2

This Example shows the preparation of the allyl halide, CF₂═CHCF₂Br.

CF₂═CHCF₂Br (b.p. 34° C.) was prepared as in Example 1 from CF₂Br₂ and CF₂═CH₂.

### Example 3

This Example shows the preparation of the symmetrical fluorinated diene alcohol, CH₂═CHCF₂C(CF₃)(OH)CF₂CH═CH₂.

Zinc powder (8.0 g, 0.12 mol) and THF(50 ml) were added to a 250 mL three neck flask fitted with stir bar, reflux condenser, addition funnel, and N₂ inlet. The equipment was protected under an N₂ blanket and the flask was cooled in ice. (CF₃CO)₂O (10.5 g. 0.05 mol) was added to flask and stirred. Then CH₂═CHCF₂Br (15.7 g, 0.1 mol) in THF (50 mL) was added drop-wise from the addition funnel over about 2 hours. The reaction mixture was stirred overnight and then evaporated in a rotovap at 25° C. and 50 mm Hg pressure. The remaining solid containing the product was evacuated at 40-50° C. and 5 mm Hg pressure. 10 g of product was separated from the solid by condensing it into a cold trap at −75° C. The GC and NMR showed pure product containing some THF.

### Example 4

This Example shows the preparation of the symmetrical fluorinated diene alcohol, CF₂═CHC(CF₃)(OH)CH═CF₂.

This compound is prepared and analyzed by the procedure described in Example 3, but from CF₂═CHBr and (CF₃CO)₂O.

### Example 5

This Example shows the preparation of the symmetrical fluorinated diene alcohol, CF₂═CHCF₂C(CF₃)(OH)CF₂CH═CF₂.

This compound is prepared and analyzed by the procedure described in Example 3, but from CF₂═CHCF₂Br and (CF₃CO)₂O.

### Example 6

This Example shows the preparation of the allyl trifluoromethyl ketone, CH₂═CHCF₂COCF₃, using a magnesium reagent.

(CF₃CO)₂O (34.2 g, 0.3 mol) was dissolved in 75 mL of anhydrous ether. The solution was then added drop-wise over 2 hours to a rapidly stirred solution of CH₂═CHCF₂MgBr (from CH₂═CHCF₂Br and Mg, 0.3 mol in 200 mL of anhydrous ether) at −78° C. The mixture was refluxed for 2 hours and then poured into a mixture of ice and excess concentrated HCl. The layers were separated and the aqueous phase was extracted with ether. The combined ether extracts were washed with saturated NaHCO₃ and dried with MgSO₄. The solvent was removed and the residue was distilled to give 40-50% of CH₂═CHCF₂COCF₃.

### Example 7

This Example shows the preparation of the allyl trifluoromethyl ketone, CH₂═CHCF₂COCF₃, using Zn powder.

CH₂═CHCF₂Br (35 g, 0.22 mol) was added to a stirred mixture of pyridine(100 mL), ethyl trifluoroacetate (24.4 g, 0.2 mol), and Zn powder (15 g, 0.2 mol) under protection of N₂. After reacting, the reaction mixture is treated as in example 6 to produce a 40-50% yield of CH₂═CHCF₂COCF₃.

### Example 8

This Example shows the preparation of the allyl trifluoromethyl ketone, CF₂═CHCF₂COCF₃, from magnesium reagent.

The compound is prepared as per the process described in Example 6 except using CF₂═CHCF₂MgBr and (CF₃CO)₂O.

### Example 9

This Example shows the preparation of the allyl trifluoromethyl ketone, CF₂═CHCF₂COCF₃, using Zn powder.

The compound is prepared as per the process described in Example 7 except using CF₂═CHCF₂Br, (CF₃CO)₂O, and zinc powder.

### Example 10

This Example shows the preparation of the asymmetrical fluorinated diene alcohol, CF₂═CHCF₂C(CF₃)(OH)CH═CH₂.

The compound is prepared as per the process described in Example 7 except using CF₂═CHCF₂COCF₃ and CH₂═CHBr.

### Example 11

This Example shows the preparation of the asymmetrical fluorinated diene alcohol, CF₂═CHCF₂C(CF₃)(OH)CH═CF₂.

The compound is prepared as per the process described in Example 7 except using CF₂═CHCF₂COCF₃ and CF₂═CHBr.

### Example 12

This Example shows the preparation of the asymmetrical fluorinated diene alcohol, CF₂═CHCF₂C(CF₃)(OH)CF₂CH═CH₂.

The compound is prepared as per the process described in Example 7 except using CF₂═CHCF₂COCF₃ and CH₂═CHCF₂Br.

### Example 13

This Example shows the preparation of the asymmetrical fluorinated diene alcohol, CH₂═CHCF₂C(CF₃)(OH)CH═CF₂.

The compound is prepared as per the process described in Example 7 except using CH₂═CHCF₂COCF₃ and CF₂═CHBr.

Having thus described a few particular embodiments of the invention, various alterations, modifications, and improvements will readily occur to those skilled in the art. Such alterations, modifications, and improvements, as are made obvious by this disclosure, are intended to be part of this description though not expressly stated herein, and are intended to be within the spirit and scope of the invention. Accordingly, the foregoing description is by way of example only, and not limiting. The invention is limited only as defined in the following claims and equivalents thereto.

What is claimed is:

1. A fluorinated diene alcohol compound comprising:
at least about 45 percent by weight of fluorine and from about 6 to about 11 carbon atoms, said compound including at least one moiety of the formula CFₓ and at least one moiety of the formula CHₓ, where each x is independently 1, 2 or 3 and where each CFₓ moiety is not directly bonded to another CFₓ moiety.

2. The compound of claim 1 having the formula (I): 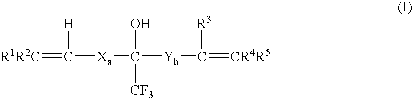
where
R¹, R², R⁴, and R⁵ are independently H or F;
R³ is H, CH₃, or CF₃;
X and Y are independently CH₂ or CF₂; and
a and b are independently 0 or 1;
provided that if X is CH₂, then at least one of R¹ or R² is fluorine and that if Y is CH₂, then at least one of R³, R⁴, or R⁵ is fluorine or CF₃.

3. The compound of claim 2 symmetrical about its —C(OH)(CF₃)— moiety.

4. The compound of claim 2 asymmetrical about its —C(OH)(CF₃)— moiety.

5. The compound of claim 2 comprising a moiety of the formula C(OH)(CF₃)(CFₓ)₂, wherein each c is independently 1 or 2.

6. The compound of claim 2 comprising a moiety of the formula C(OH)(CF₃)(CH_{d})₂, wherein each d is independently 1 or 2.

7. The compound of claim 2 comprising a moiety of the formula C(OH)(CF₃)(CF_{c})(CH_{d}), wherein c is 1 or 2 and d is 1 or 2.

8. The compound of claim 3 selected from the group consisting of CF₂═CHC(CF₃)(OH)CH═CF₂, CF₂═CHCF₂C(CF₃)(OH)CF₂CH═CF₂, and CH₂═CHCF₂C(CF₃)(OH)CF₂CH═CH₂.

9. The compound of claim 4 selected from the group consisting of CF₂═CHCF₂C(CF₃)(OH)CH═CH₂, CF₂═CHCF₂C(CF₃)(OH)CH═CF₂, CF₂═CHCF₂C(CF₃)(OH)CF₂CH═CH₂, CH₂═CHCF₂C(CF₃)(OH)CH═CF₂, CH₂═CHCF₂C(CF₃)(OH)C(CH₃)═CF₂, and CH₂═CHCF₂C(CF₃)(OH)CH═CH₂.

10. The compound of claim 1 comprising from about 45 to about 70 weight percent of fluorine.

11. A method of synthesizing fluorinated diene alcohol comprising reacting at least one C₂-C₃ alkene halide with at least one reactant of the formula (II): 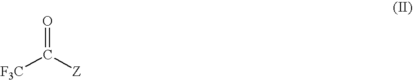
where
Z is OH, OR⁶, OC(O)R⁷, or a halogen,
R⁶ is a C₁-C₆ branched or straight chain alkyl, and
R⁷ is a C₁-C₆ branched or straight chain alkyl or fluoroalkyl,
to produce a C₆-C₁₁ fluorinated diene alcohol.

12. The method of claim 11 wherein said fluorinated diene alcohol is a compound according to claim 1.

13. The method of claim 11 wherein said fluorinated diene alcohol is a compound according to claim 2.

14. The method of claim 13 wherein said at least one C₂-C₃ alkene halide comprises vinyl halide comprising at least one H atom and at least one atom selected from the group consisting of chlorine, bromine, and iodine.

15. The method of claim 13 wherein said at least one C₂-C₃ alkene halide comprises allyl halide comprising at least one H atom and at least one atom selected from the group consisting of chlorine, bromine, and iodine.

16. The method of claim 14 wherein said at least one C₂-C₃ alkene halide further comprises at least one fluorine atom.

17. The method of claims 15 wherein said at least one of said C₂-C₃ alkene halide further comprises at least one fluorine atom.

18. The method of claim 13 wherein said at least one C₂-C₃ alkene halide is selected from the group consisting of CH₂═CHCH₂Cl, CH₂═CHCF₂Br, CF₂═CHCF₂Br, CH₂═CHBr, CF₂═CHBr, CF₂═C(CH₃)Br, and combinations of these.

19. The method of claim 1 wherein said reaction is carried out in the presence of a catalyst.

20. The method of claim 19 wherein said catalyst comprises a metal-containing catalyst.

21. A method of synthesizing an asymmetrical fluorinated diene alcohol comprising:
(a) reacting at least a first C₂-C₃ alkene halide with at least one reactant selected from the group consisting of trifluoroacetic acid, ethyl trifluoroacetate, and trifluoroacetic anhydride to form at least one fluorinated allyl trifluoromethyl ketone; and
(b) reacting at least one allyl trifluoromethyl ketone of step (a) with a second C₂-C₃ alkene halide to form an asymmetrical fluorinated diene alcohol.

22. The method of claim 21 wherein said reacting step (a) is conducted at least in part in the presence of metal catalyst.

23. The method of claim 21 wherein said metal catalyst comprises magnesium, cadmium, or zinc.

24. The method of claim 21 wherein said reacting step (b) is conducted at least in part in the presence of zinc.

25. The method of claim 21 wherein said asymmetrical fluorinated diene alcohol is selected from the group consisting of CF₂═CHCF₂C(CF₃)(OH)CH═CH₂, CF₂═CHCF₂C(CF₃)(OH)CH═CF₂, CF₂═CHCF₂C(CF₃)(OH)CF₂CH═CH₂, CH₂═CHCF₂C(CF₃)(OH)CH═CF₂, CH₂═CHCF₂C(CF₃)(OH)C(CH₃)═CF₂, and CH₂═CHCF₂C(CF₃)(OH)CH═CH₂.

26. A method of synthesizing a symmetrical diene alcohol comprising reacting at least one first C₂-C₃ alkene halide and at least one reactant selected from the group consisting of trifluoroacetic ester and trifluoroacetic anhydride to form a symmetrical fluorinated diene alcohol.

27. The method of claim 26 wherein said symmetrical diene alcohol is selected from the group consisting of CF₂═CHC(CF₃)(OH)CH═CF₂, CF₂═CHCF₂C(CF₃)(OH)CF₂CH═CF₂, and CH₂═CHCF₂C(CF₃)(OH)CF₂CH═CH₂.
